(19) United States
(12) Patent Application Publication
(10) Pub. No.: US 2009/0191135 A1
(43) Pub. Date: Jul. 30, 2009

(54) METHODS FOR THE DISPERSION OF WATER-SOLUABLE OR HYDROPHILIC SUBSTANCES IN A SUPERCRITICALLY PRESSURIZED FLUID

(75) Inventors: Joel RICHARD, St. Barthelemy d'anjou (FR); Frantz Deschamps, Nancy (FR); Patrick Lacroix-Desmazes, Montpellier (FR); Bernard Boutevin, Montpellier (FR)

Correspondence Address: FOLEY AND LARDNER LLP;SUITE 500, 3000 K STREET NW, WASHINGTON, DC, 20007, US

(73) Assignee: ETHYPHARM CENTRE NATIONAL DE LA RECHERCHE SCIENTIFIQUE (CNRS

(21) Appl. No.: 12/355,696

(22) Filed: Jan. 16, 2009

Related U.S. Application Data
(62) Division of application No. 10/554,281, filed on Apr. 13, 2006, now abandoned.

(30) Foreign Application Priority Data
Apr. 25, 2003 (FR) 0305108

(51) Int. Cl. A61K 9/12

(52) U.S. Cl. 424/45

(57) ABSTRACT

A method for encapsulating an active principle by dispersing the active principle in a supercritical fluid by adding a surfactant to the fluid. The surfactant is a block copolymer having at least one CO₂-philic block and at least one nonionic hydrophilic block.

The present invention relates to a process for the dispersion of water-soluble or hydrophilic substances in a supercritical fluid.

Many industrial processes, in particular in the pharmaceutical field, use dispersions of solid particles in an organic solvent or emulsions of an aqueous medium in an organic solvent. Mention will be made in particular of processes for encapsulation by coacervation of a coating agent dissolved in an organic solvent, bringing about the deposition of this coating agent at the surface of the particles suspended in the organic solution, and emulsion/solvent extraction or double emulsion processes.

These processes involve large amounts of organic solvents, such as halogenated solvents, and are accompanied by high environmental costs. In the pharmaceutical field, these processes require the implementation of methods for monitoring the residual solvents and optionally for reducing the content of residual solvent in pharmaceutical formulations. The trend is to reduce or even eliminate these solvents in processes intended for the production of pharmaceutical formulations. Finally, it is necessary to add that many pharmaceutical active principles, such as, for example, therapeutic proteins, can be denatured by contact with an organic solvent.

Carbon dioxide (CO₂) at supercritical pressure is finding increasing applications in numerous processes where it advantageously substitutes for solvents. In contrast to the dispersions in an organic solvent, the unique properties of CO₂ at supercritical pressure make it possible, in the case of dispersions in CO₂ at supercritical pressure, to separate the phases by simple decompression of the medium, thus resulting in easy recovery of the substances previously dispersed. These substances are furthermore devoid of residual organic solvent. The formation of dispersions in CO₂ at supercritical pressure must thus offer advantageous possibilities of substituting for organic solvents in major industrial processes, such as separations, reactions or processes for the formation of specific materials and in particular processes resulting in pharmaceutical formulations in the form of powders.

The Applicant has endeavored to develop alternative processes free from organic solvent which make use of the advantageous properties of CO₂ at supercritical pressure. The term “CO₂ at supercritical pressure” denotes here CO₂ brought to a pressure in the vicinity of or greater than its critical pressure (7.38 MPa). It can then be either in the liquid state or in the supercritical state. Thus, patent EP 0 784 506 B1 has as subject matter a process for the preparation of microparticles composed of solid particles which are insoluble in CO₂ at supercritical pressure coated with a coating agent which is soluble in CO₂ at supercritical pressure. The key stage of this process is the deposition of the coating agent on the particles in suspension in the supercritical fluid by controlled modification of the temperature and/or of the pressure of the supercritical fluid. The particles to be coated are conventionally dispersed in the supercritical fluid by stirring. The improvements made to the abovementioned process have led the Applicant to look for means for improving the properties of dispersions of particles in the supercritical fluid, with the main object of stabilizing the dispersion and thus of reducing the phenomena of agglomeration of the particles brought about by a difficulty in dispersing hydrophilic or water-soluble particles in a homogeneous and stable manner in the fluid at supercritical pressure.

Dispersions of solid particles are regarded as stable if, over time and under the effect of the various interactions which prevail in the dispersed system, the particles remain isolated and do not form reversible or irreversible aggregates and the concentration of particles remains homogeneous at every point in the dispersing medium, that is to say no phase separation appears. A loss in stability generally results first in the formation of aggregates of particles (flocculation, coagulation), related to the appearance of attractive forces between the particles, and subsequently in a phase separation (sedimentation or creaming), due to the difference in density between the large aggregates thus formed and the dispersing medium (syneresis effect). The mechanisms for stabilizing dispersions of solid particles are known to a person skilled in the art and are described in the reference papers by R. H. Ottewill (Journal of Colloid & Interface Science, Vol. 58, No. 2, pp. 357-373, February 1977) and by J. Th. G. Overbeek (Journal of Colloid & Interface Science, Vol. 58, No. 2, pp. 408-422, February 1977). They involve electrostatic interactions, when the particles carry electric charges at their surface which bring about repulsions between them and prevent the formation of aggregates. In the case of particles which do not carry electric charges, steric stabilization of the dispersions can be obtained by introducing neutral (nonionic) polymer chains of well chosen length at the surface of the particles. These chains can either be attached chemically, on the one hand, or, on the other hand, can be adsorbed at the surface or physically implanted in the surface layer of the dispersed solid particles. They must also exhibit a part which is soluble in a good solvent in the dispersing phase. These chains thus exhibit a degree of surface-active or surfactant nature. The notion of steric stabilization was initially introduced by W. Heller and T. L. Pugh in 1954 (Journal of Chemical Physics, Vol. 22, p. 1778, 1954). The stabilization is generated, on the one hand, by the repulsive forces related to the enthalpy of mixing of the stabilizing chains in a good solvent and, on the other hand, by the elasticity of the polymer layer formed at the surface of the solid particles (entropic origin). The relative contributions of these two mechanisms depend mainly on the temperature, on the good or poor solvating nature of the dispersing phase and on the density and thickness of the stabilizing layer of polymer. A complete review of steric stabilization mechanisms is given in the reference paper by D. H. Napper (Journal of Colloid & Interface Science, Vol. 58, No. 2, pp. 390-407, 1977). An example of the dispersion of hydrophobic (polyacrylonitrile) particles sterically stabilized in a hydrophobic organic solvent (toluene) by polystyrene chains adsorbed at their surface has been given by A. Doroszwski and R. Lamboume (Journal of Colloid & Interface Science, Vol. 43, p. 97, 1973). Several examples of dispersions of solid hydrophilic (poly(12-hydroxystearic acid), poly(oxyethylene)) particles in nonaqueous organic solvents (n-heptane, methanol) have been given by D. H. Napper (Journal of Colloid & Interface Science, Vol. 58, No. 2, pp. 390-407, 1977). Dense and hydrophilic inorganic particles of iron oxide have also been dispersed and sterically stabilized in cyclohexanone by polyamide chains adsorbed at their surface (T. Sato, Journal of Coatings Technology, Vol. 65, No. 825, pp. 113-121, 1993).

The majority of commercial surfactants are insoluble in CO₂ at supercritical pressure (Consani et al., Journal of Supercritical Fluids, 3 (1990), 51-65) and thus cannot be used to stabilize dispersions in CO₂ at supercritical pressure. However, a few surfactants soluble in CO₂ at supercritical pressure are described in the literature. It is well known that the majority of polymers are insoluble in CO₂ at supercritical pressure at temperatures compatible with processes intended for the production of pharmaceutical formulations (Kirby C. F. and McHugh, Chemical Review, 99 (1999), 565-602). A few families of CO₂-philic polymers are substantially soluble in supercritical CO₂. These polymers are predominantly fluoropolymers (Newman D. A. et al., Journal of Supercritical Fluids, 6 (1993), 305-210), such as poly(fluoroether)ₛ, poly(fluoroacrylate)s or poly(fluoromethacrylate)s, silicones, such as poly(dimethylsiloxane)s (Hoefling et al., Journal of Supercritical Fluids, 6 (1993), 164-171) or poly(ether carbonate)s (Sarbu et al., Nature, 405 (2000), 165-167).

CO₂-philic polymers have been employed to synthesize surfactants which can be used in CO₂ at supercritical pressure. They are either CO₂-philic polymers functionalized so as to increase their affinity for the substances to be dispersed or copolymers comprising chain members chosen from the polymers soluble in CO₂ (CO₂-soluble polymers) described previously. The main applications of these surfactants are processes for polymerization in dispersion in supercritical CO₂, where these surfactants make possible steric stabilization of the particles of polymers being formed. The chain members intended to interact with the particles of polymers being formed are hydrophobic chain members, such as, for example, polystyrenes, poly(vinyl acetate)s or poly(methyl methacrylate)s.

These compounds are all surfactants of the CO₂ philic/hydrophobic type. They thus cannot be used to stabilize dispersions of liquid or solid hydrophilic substances in CO₂ at supercritical pressure as they are devoid of a hydrophilic part which can interact with hydrophilic materials and become attached to their surface.

CO₂-philic surfactants with an ionizable head have been synthesized and evaluated for the formation of emulsions of water in supercritical CO₂. These compounds are composed of a CO₂-philic part, such as fluoropolymers or poly(siloxane)s, and of a short ionized head which can interact electrostatically with the aqueous phase to be dispersed. These surfactants have, in some specific cases, made possible the formation of emulsions or of microemulsions of water in supercritical CO₂ (Hoefling T. A. et al., Journal of Physical Chemistry, 95 (1991), 7127-7129). However, they cannot be used to stabilize dispersions of solid hydrophilic particles in supercritical CO₂, that is to say in a nonaqueous medium. This is because the stabilization of such dispersions requires a strong interaction of the surfactant with the surface of the particles to be dispersed, making possible the permanent attachment of the polymer surfactants at their surface. It is beyond the scope of the surfactants carrying a short ionizable polar head, which is furthermore nonionized in a nonaqueous medium.

Attempts at the steric stabilization of inorganic substances, such as particles of hydrophilic silica, have been published (L. Calvo et al., Journal of Supercritical Fluids, 16 (2000), 247-260). It is demonstrated, in this study, that the use of surfactants soluble in supercritical CO₂, such as a perfluoropolyether carrying an ammonium carboxylate short ionic head, or a polystyrene-poly(1,1-dihydroperfluorooctyl acrylate) copolymer, or else a poly(methyl methacrylate-co-hydroxyethyl methacrylate) copolymer grafted with polyfluoropolyether polymers, does not make possible the steric stabilization of particles of hydrophilic silica with a size of greater than 1.2 μm in supercritical CO₂ starting from a dispersion of polydispersed particles (broad particle size distribution) of silica with a mean size of 3 μm, 90% of which have a size of greater than 1.5 μm. While a suspension of the particles with a smaller diameter, of between 0.7 and 1.2 μm, is stabilized over a short period of time, only a minute fraction, less than 2.5%, of the mass of silica particles to be dispersed can be stabilized by these surfactants. These surfactants, nevertheless soluble in supercritical CO₂ and capable of stabilizing dispersions of hydrophobic substances in supercritical CO₂, thus do not make it possible to efficiently stabilize dispersions of particles of hydrophilic silica in supercritical CO₂.

None of the CO₂-philic surfactants currently known thus makes it possible to prepare stable dispersions of water-soluble or hydrophilic solid substances in CO₂ at supercritical pressure.

### SUMMARY OF THE INVENTION

The inventors have found, surprisingly, that the use of block copolymers composed of at least one CO₂-philic block and of at least one nonionic hydrophilic block, of sui table composition and of sui table molar mass, makes it possible to prepare stable dispersions of hydrophilic substances in CO₂ at supercritical pressure.

### DETAILED DESCRIPTION OF THE PREFERRED EMBODIMENTS

The copolymers are chosen in particular from the group consisting of diblock copolymers and triblock copolymers, preferably diblock copolymers.

The triblock copolymers can correspond either to the formula (1)

hydrophilic/CO₂-philic/hydrophilic (1),

or to the formula (2)

CO₂-philic/hydrophilic/CO₂-philic (2),

in which, respectively, the hydrophilic or CO₂-philic groups can be identical or different.

The present invention relates to the use of surfactants specifically designed to prepare stable dispersions of water-soluble or hydrophilic substances in a fluid at supercritical pressure. These substances are in the solid or liquid state under the temperature and pressure conditions of the process and are composed of materials which are predominantly water-soluble or hydrophilic and insoluble in CO₂ at supercritical pressure.

The Applicant has designed and synthesized novel block copolymers comprising at least one CO₂-philic block and at least one nonionic hydrophilic block. These copolymers have proved to be capable of stabilizing dispersions of hydrophilic solid particles in CO₂ at supercritical pressure. They can be employed in particular for improving the encapsulation process disclosed in patent EP 0 784 506 81. The block copolymers are used both as agents for stabilizing the dispersion of hydrophilic particles and/or as coating agent. These copolymers can also be employed as surfactants for stabilizing a dispersion of particles to be coated, in conjunction with coating agents conventionally used in the abovementioned process, such as lipids, waxes, polymers or any compound which is sufficiently soluble in CO₂ at supercritical pressure and which can be used in the implementation of the above-mentioned process.

It has been discovered, in the context of the present invention, that these specific surfactants can also be used for the formation and the steric stabilization of emulsions of water or of aqueous solutions in CO₂ at supercritical pressure.

The process which is a subject matter of the invention is particularly suitable for the production of pharmaceutical formulations, the processes for the preparation of which comprise a stage of dispersing water-soluble or hydrophilic substances in a dispersing medium composed of CO₂ at supercritical pressure.

The dispersing medium is CO₂ brought to a pressure in the region of or greater than the critical pressure (7.38 MPa), preferably between 5 MPa and a pressure of 70 MPa, more preferably still between the critical pressure and a pressure of 30 MPa. The temperature is between 0° C. and 100° C. and preferably between 15° C. and 60° C.

The dispersing medium can optionally comprise an entrainer. Within the meaning of the present invention, an entrainer is defined as a substance deliberately added in small amounts to the supercritical fluid so as to increase the solubility of one or more substance(s) in said supercritical fluid. When such an entrainer is present in a small amount in the supercritical fluid (approximately <5%), this amount is so low that it is essentially without effect on the conditions necessary for the entry into the supercritical state of the main component of the supercritical fluid but greatly increases the solubility of the polymer material. Use may be made, as entrainer, by way of examples, of ketones, alcohols, esters and chlorinated solvents or other organic solvents and plasticizers. Such entrainers are particularly used when the polymer is not very soluble in CO₂ at supercritical pressure.

In the case of dispersions of substances which are solid under the conditions of the process, the particles to be dispersed are composed of a hydrophilic or water-soluble material which is insoluble in the fluid at supercritical pressure, preferably particles comprising an active principle.

Water-soluble or hydrophilic substances or substances which are included in predominantly water-soluble or hydrophilic particles can be dispersed in CO₂ at supercritical pressure by the process which is a subject matter of the invention. Hydrophilic active principles or active principles dissolved or dispersed in a water-soluble or hydrophilic matrix are particularly employed in the formation of dispersions by the process of the invention.

Mention may nonexhaustively be made, among active principles, of (i) pharmaceuticals, in particular analgesics, antipyretics, aspirin and its derivatives, antibiotics, anti-inflammatories, antiulceratives, antihypertensives, neuroleptics, antidepressants, oligonucleotides exhibiting a therapeutic activity, peptides exhibiting a therapeutic activity and proteins exhibiting a therapeutic activity, (ii) cosmetics, in particular self-tanning agents and UV stabilizers, and (iii) foodstuffs, such as, for example, vitamins.

The therapeutic proteins or peptides used in these formulations are preferably chosen from the protein corresponding to parathyroid hormone (PTH), growth hormone (GH), α-, β- or γ-interferons, α- or β-erythropoietin (EPO), granulocyte colony-stimulating factor (GCSF), granulocyte-macrophage colony-stimulating factor (GMCSF), vasoactive intestinal peptide (VIP), thyrotopin-releasing hormone (TRH), arginine vasopressin (AVP), angiotensin, insulin, somatotropin, tissue plasminogen activator, clotting factors VIII and IX, glucosylceramidase, lenograstim, molgramostim, filgrastim, interleukins, dornase alfa, PEG-L-asparaginase, PEG-adenosine deaminase, hirudin, eptacog alfa, nerve growth factors, luteinizing hormone-releasing hormone (LHRH), its derivatives and its analogs, somatostatin and its derivatives, triptorelin, bombesin, calcitonin, gastrin-releasing peptide, growth hormone-releasing factor and amylin.

The particles comprising an active principle which result from a process for precipitation or crystallization using organic solvents or fluids at supercritical pressure, from a lyophilization process, from a nebulization process, from a drying process or from a milling process which are capable of being dispersed in the fluid at supercritical pressure are preferably composed of a solid matrix comprising the active principle, in particular a therapeutic protein or peptide, and optionally excipients, such as fillers, protein stabilizers, cryoprotectants and lyoprotectants. Mention may nonexhaustively be made, among the excipients, of phosphate salts and other ionic compounds, sugars, polyols, filler proteins, such as albumins, surfactants and any other substance used to stabilize solid formulations formed of therapeutic proteins, existing or to come. These particles comprising active principles, in particular proteins or peptides, are predominantly hydrophilic and difficult to disperse in CO₂ at supercritical pressure.

The size of the particles dispersed by the process which is a subject matter of the invention is between 0.05 μm and 800 μm and preferably between 0.1 μm and 100 μm.

The copolymers used in the process of the invention are block copolymers composed of a CO₂-philic block and of a nonionic hydrophilic block. The nature of the two blocks and their molar ratio can be altered and make possible fine adjustment of the properties for stabilization of dispersions of hydrophilic solids or liquids which are insoluble in CO₂ at supercritical pressure.

The CO₂-philic block of the block copolymers is advantageously chosen from polymers which are soluble in CO₂ at supercritical pressure.

The block copolymers are advantageously copolymers which are soluble in CO₂ at supercritical pressure. The minimum solubility of the block copolymers according to the invention is 0.05% w/w and preferably 0.2% w/w at least one defined temperature which is between 0° C. and 100° C., preferably at least one defined temperature which is between 15° C. and 60° C., and at least one defined pressure which is greater than the critical pressure of CO₂, preferably less than 70 MPa and more preferably still less than 30 MPa.

The molar mass of the block copolymers which are subject matters of the invention and the molar ratio of the two CO₂-philic and hydrophilic blocks have to be chosen so as to give the copolymer a sufficient solubility in CO₂ at supercritical pressure and a solubility which is advantageous for the process which is a subject matter of the invention, under temperature and pressure conditions which are technically and economically advantageous and which make possible the use of unstable substances, such as pharmaceutical active principles.

The molar mass and the molar ratio are also chosen in order to obtain the desired properties for stabilization of dispersions. With regard to these considerations of solubility and of stabilization of the dispersions, it has been found, in the context of the present invention, that the number-average molar mass (Mn) of the copolymer has to be chosen between 1000 and 200 000 g/mol and more particularly between 4000 and 50 000 g/mol. The number-average molar mass of the hydrophilic block is between 500 and 20 000 g/mol and preferably between 1000 and 10 000 g/mol. The ratio by weight of the CO₂-philic block to the hydrophilic block, defined as the ratio of the number-average molar masses, is between 1 and 50 and preferably between 1 and 20.

Mention may in particular be made, among the polymers soluble in CO₂ used in the synthesis of the block copolymers, of fluoropolymers and poly(siloxane)s. Among the fluoropolymers, the polymers used are advantageously chosen from poly(fluoroether)s, poly(fluoroalkyl methacrylate)s and poly(fluoroalkyl acrylate)s and particularly poly(1,1-dihydroperfluoro octyl acrylate)s or PFOA and poly(1,1,2,2-tetrahydroperfluorodecyl acrylate)s or PFDA.

The nonionic hydrophilic block is advantageously chosen from biocompatible hydrophilic polymers, in particular from the group consisting of polysaccharides, hydrophilic cellulose polymers, poly(vinyl alcohol), polyols, and ethylene oxide homo- and copolymers. Preferably, the hydrophilic block is a poly(ethylene oxide) or PEO.

The block copolymers used according to the process of the invention are advantageously block copolymers composed of a poly(1,1,2,2-tetrahydroperfluorodecyl acrylate) block and of a poly(ethylene oxide) block or are block copolymers of the PEO-b-PFDA type.

Mention may be made, among the triblock copolymers according to the invention, without implied limitation, of PFDA-b-PEO-b-PFDA triblock copolymers and PEO-b-PFDA-b-PEO triblock copolymers.

These copolymers are synthesized by any suitable synthetic route which offers access to the composition desired for the copolymer in terms of nature of the CO₂ philic and hydrophilic blocks, of molar mass and of molar ratio of the two blocks. The synthesis is preferably carried out by a conventional radical telomerization process.

The copolymers can be purified after synthesis by any process commonly used for this purpose, such as precipitations or chromatographic purifications, and preferably by extraction or fractionation using CO₂ at supercritical pressure due to their intrinsic solubility in this medium. This extraction uses conventional batchwise or continuous devices for extraction and fractionation by a supercritical fluid.

Hydrophilic substances are dispersed by any means commonly used to bring about the dispersion of solid or liquid substances in a fluid. In the case of dispersions of solid particles, the dispersing means preferred for the present invention are stirring by use of a rotating stirrer and fluidization by use of a bed fluidized with CO₂ at supercritical pressure.

The use of these block copolymers is particularly beneficial in processes for coating or modifying substances dispersed in CO₂ at supercritical pressure. This is because these processes can be improved by stabilization of the dispersion, limiting phenomena of aggregation.

The invention is thus of particular use in processes for the encapsulation of particles of pharmaceutical interest which are intended, for example, to improve the rheological behavior of powders, to improve the stability of the formulation, to modify their surface properties or to confer controlled release properties thereon.

The present invention is particularly suitable for processes for coating particles dispersed in CO₂ at supercritical pressure, such as the process disclosed in patent EP 0 784 506 B1, where a controlled modification of the temperature and/or of the pressure of CO₂ at supercritical pressure makes possible the controlled deposition of a coating agent which is soluble in CO₂ at supercritical pressure. The block copolymers are then used mainly as surfactants, making possible the stabilization of the dispersion, and optionally as coating agents, alone or as a mixture with other coating agents.

The present invention also makes it possible to form and to stabilize emulsions of water or of aqueous solutions in CO₂ at supercritical pressure. Processes using a stage requiring the stabilization of emulsions of aqueous solutions in an organic solvent, such as, for example, the formation of hydrogels in inverse dispersion or emulsion, can advantageously benefit from the present invention, which makes it possible to replace organic solvents with carbon dioxide.

Another subject matter of the present invention is block copolymers composed of a CO₂-philic block and of a biocompatible nonionic hydrophilic block.

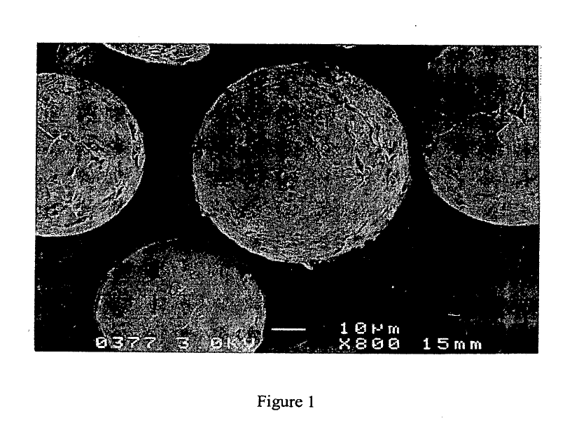

The following examples and FIGS. 1 to 6 and 8 to 10 illustrate the invention.

Examples 7 and 11 are comparative examples.

### BRIEF DESCRIPTION OF THE DRAWINGS

The appearance by scanning electron microscopy of the particles which have been subjected to an encapsulation process as described in example 1 is represented in FIG. 1.

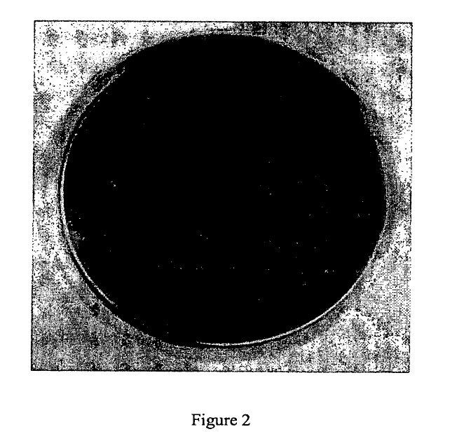

A photograph of an emulsion of an aqueous solution of amaranth in supercritical CO₂ at a temperature of 40° C. and a pressure of 30 MPa, obtained in the presence of PEO-b-PFDA block copolymers according to the process of the invention, is represented in FIG. 2.

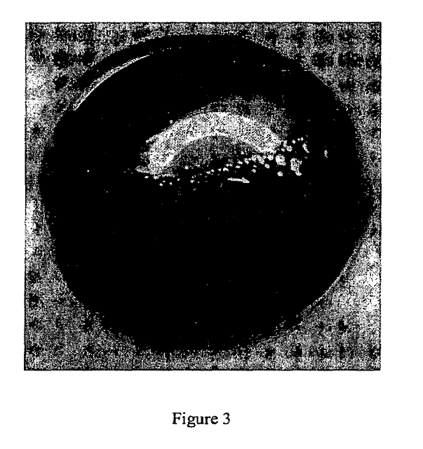

A photograph of a mixture of an aqueous solution of amaranth and of supercritical CO₂ at a temperature of 40° C. and a pressure of 30 MPa, with stirring but in the absence of PEO-b-PFDA block copolymer, is represented in FIG. 3.

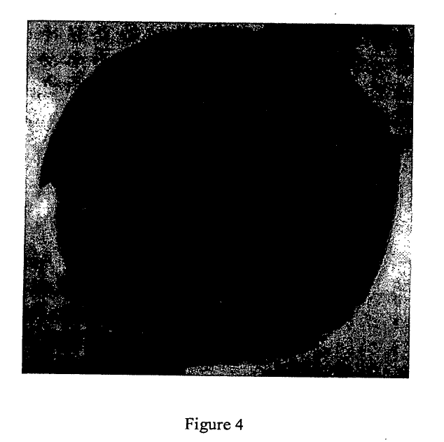

A photograph of an emulsion of an aqueous solution of amaranth in liquid CO₂ at a temperature of 20° C. and a pressure of 30 MPa, obtained according to the process of the invention, is represented in FIG. 4.

### EXAMPLE 1

### Synthesis of a PEO-b-PFDA Block Copolymer

This example describes the synthesis of a block copolymer composed of a poly(ethylene oxide) block with a number-average molar mass (Mn) of 2000 g/mol (PEO 2000) and of a poly(1,1,2,2-tetrahydroperfluorodecyl acrylate) (PFDA) block, i.e. a diblock copolymer of the PEO-b-PFDA type. The synthesis is carried out by conventional radical telomerization.

In a first stage, a macromolecular telogen (I) is synthesized from PEO 2000.

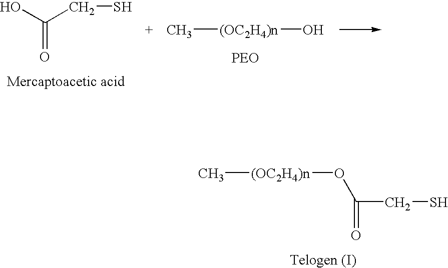

In a second stage, the telomerization of the fluoroacrylate 1,1,2,2-tetrahydroperfluorodecyl acrylate (FDA) is carried out in the presence of the telogen (I) to form the block copolymer.

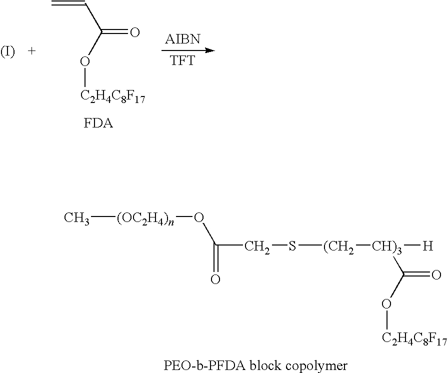

### 1.1. Synthesis of the Macromolecular Telogen (I)

50.03 g (25 mmol) of poly(ethylene oxide) monomethyl ether (Aldrich, Mₙ=2000 g/mol) and 705.7 g of benzene are incorporated in a 1 liter three-necked flask equipped with a Dean and Stark apparatus, a reflux condenser and a magnetic flea. The polymer is dried by azeotropic entrainment at reflux (set temperature of 100° C.) for 5 hours. Subsequently, 12.0243 g (127.9 mmol) of thioglycolic acid (Acros, 98%, M=92.11 g/mol) and 127 mg (0.658 mmol) of para-toluene-sulfonic acid monohydrate (Aldrich, 98.5%) are added. The azeotropic entrainment is continued for 24 hours, the water formed accumulated in the Dean and Stark apparatus being regularly withdrawn.

At the end of the reaction, the medium is brought back to ambient temperature, filtered through a sintered glass No. 4, in order to remove a few insoluble materials, and then concentrated on a rotary evaporator at 40° C. 59.97 g of a viscous product are recovered, which product crystallizes at ambient temperature. It is dissolved in 31 g of chloroform (very viscous solution). This solution is precipitated, with stirring, from 4 liters of ethyl ether cooled beforehand in a freezer. Filtration is carried out through a sintered glass No. 4 and the cake is washed in the sintered glass with a further 1 liter of ethyl ether at ambient temperature. 55.01 g of white powder are recovered, which powder is dried under vacuum (2×10⁻² mmHg) at 35-40° C. for 2 hours. 45.87 g of white powder are finally obtained (yield=88%).

### 1.2. Synthesis of a PEO-b-PFDA Copolymer

The fluoroacrylate monomer 1,1,2,2-tetrahydroperfluorodecyl acrylate (FDA) is distilled under vacuum (P=3×10⁻² mmHg, B.p.=70-71° C.). The 2,2′-azobisisobutyronitrile (AIBN) (Fluka, 98%) is recrystallized from methanol. The solvent, trifluorotoluene (Lancaster, 99%) (TFT), is distilled under partial pressure (20 mmHg). The polymerization is carried out under argon in a two-necked 100 ml Schlenk reactor, closed with 2 turn-over closures, with magnetic stirring (magnetic flea).

The fluoromonomer, placed in a sample tube, is deoxygenated by bubbling with argon (for 45 min). The transfer agent and the initiator are placed in solution in the trifluorotoluene in the Schlenk reactor (slight heating with a heat gun to dissolve the telogen). The solution is deoxygenated by bubbling with argon (45 min). The monomer is added to the Schlenk reactor using a glass syringe while flushing with argon. The Schlenk reactor is closed and is then placed in an oil bath at the desired temperature (65° C. or 80° C.). The initial medium, cloudy after the addition of the monomer, becomes clear with heating and with stirring after approximately 1 minute. Samples are withdrawn through the septum with a glass syringe, the progress of the reaction is monitored by ¹H NMR and the polymerization is halted by addition of hydroquinone. The final product is precipitated from ethyl ether (large excess), dried under vacuum at 30-35° C. and then stored in a cool place.

A first copolymer, referred to as P39, is synthesized with the following conditions:

FDA: w=10.0522 g
TFT: w=40.0 g (distilled)
AIBN: w=4.9 mg
Telogen (I): w=3.3194 g

### T=65° C.

tₒ+21 h 50: halting of the reaction

Due to its nonsolubility in conventional solvents, the copolymer cannot be analyzed unambiguously by steric exclusion chromatography. Analyses carried out by ¹H NMR (after dissolution in a TFT/Freon 113 mixture) and by elemental analysis make possible the determination of the molar ratio of the CO₂-philic component (FDA) to the hydrophilic component (PEO). In view of the fact that the reaction product may comprise PEO homopolymers, this ratio is the lower limit of the mean ratio of the CO₂-philic components to the hydrophilic components of the block copolymer formed during the synthesis. The analyses show that the reaction product is composed predominantly of a PEO-b-PFDA diblock copolymer composed of a PEO hydrophilic block with a number-average molar mass (Mn) of 2090 g/mol originating from the telogen (I) and of a PFDA CO₂-philic block with a number-average molar mass of equal to or greater than 4869 g/mol. The ratio of the CO₂-philic and hydrophilic blocks, calculated as the ratio of the molar mass Mn of the PFDA CO₂-philic block to the molar mass Mn of the PEO hydrophilic block, is thus, for this polymer P39, equal to or greater than 2.4.

### EXAMPLE 2

### Synthesis of a Series of PEO-b-PFDA Block Copolymers

A first series of PEO-b-PFDA block copolymers, referred to as P43, P44, P49, P50 and P52, based on the telogen (I) derived from PEO 2000, was synthesized so as to obtain a range of block copolymers with increasing CO₂-phile to hydrophile ratios by weight. The composition of the copolymer is adjusted by varying the weight of the FDA monomer and the weight of the telogen used in the synthesis. A second series of PEO-b-PFDA block copolymers, obtained by conventional radical telomerization from a telogen derived from a PEO with a molar mass Mn of 5000 g/mol by a synthetic route analogous to that described in example 1, was also synthesized. They are referred to as P45 and P48. The mean lower value of the CO₂-phile to hydrophile ratio by weight of these copolymers, as described in example 1, is presented in table 1.

TABLE 1

| | Lower limit of the |
|---|---|
| Copolymer | PFDA/PEO ratio |
| P43 | 6.4 |
| P44 | 7.2 |
| P49 | 37.8 |
| P50 | 28 |
| P52 | 18.9 |
| P45 | 1.2 |
| P48 | 10.2 |

### EXAMPLE 3

### Determination of the Solubility of the Copolymers in CO₂ at Supercritical Pressure

The solubility of the copolymers described in examples 1 and 2 is evaluated using a device disclosed in detail in patent EP 0 784 506 B1. This device is composed of a reactor with a maximum operating pressure of 30 MPa and with a volume of one liter equipped with a magnetically driven rotary stirring device. The reactor is equipped with a water circulation jacket for regulating the temperature. A known weight of copolymer is introduced into a preweighed paper filter with a porosity of 2 μm. The filter is then folded up and integrally attached to the upper part of the drive shaft of the stirring device, so as to provide mechanical confinement of the copolymer in this filter. The assembly is placed in the reactor. The stirring rate is set at 460 revolutions per minute. Carbon dioxide is introduced into the reactor up to a pressure 20 MPa at a temperature of 45° C. The copolymer is extracted under these temperature and pressure conditions for 1 hour. The temperature of the reactor is then reduced to approximately 23° C. over approximately 30 minutes. The reactor is subsequently gradually brought to atmospheric pressure by opening a micrometric valve which allows the carbon dioxide to move towards a vent line at atmospheric pressure. After opening the reactor, the filter is recovered and weighed, so as to calculate the weight of the fraction dissolved in CO₂ at supercritical pressure. The results of the solubility measurements for the copolymers described in examples 1 and 2 are combined in table 2.

TABLE 2
Fraction dissolved at 45° C. and 20 MPa

| Copolymer | Sample(mg) | Fraction dissolved(%) |
|---|---|---|
| P39 | 502.72 | 66.3 |
| P43 | 971.28 | 85.3 |
| P44 | 998.61 | 90.4 |
| P52 | 982.92 | 94.1 |
| P50 | 996.38 | 76 |
| P49 | 974.44 | 92.4 |
| P45 | 988.06 | 69.2 |
| P48 | 976.54 | 726 |

This device for measuring the solubility in CO₂ at supercritical pressure is routinely used by the Applicant. Due to the fact that, during the reduction in solvating power of the fluid, a portion of the dissolved coating agent is deposited on the surfaces of the filter, an extracted weight of the order of 90% is commonly observed for compounds known as completely soluble in the fluid at supercritical pressure. The copolymers synthesized in examples 1 and 2 are thus soluble in CO₂ at supercritical pressure.

### EXAMPLE 4

### Cloud Point Curve of a Copolymer

In order to quantify the solubility of the PEO-b-PFDA block copolymers in CO₂ at supercritical pressure, the cloud point curve of the fraction extracted from the copolymer P44 as obtained in example 3 was drawn up using a variable-volume optical cell.

The cloud point pressures at various temperatures and at a constant fraction by weight of copolymer of 1.2% with respect to the weight of CO₂ are presented in table 3. At a pressure greater than the cloud point pressure, it is observed visually that the sample is completely soluble. At a pressure below the cloud point pressure, cloudiness is observed visually as a result of the precipitation of the copolymer. These results confirm the solubility of the copolymer in CO₂ at supercritical pressure.

TABLE 3

| Temperature(° C.) | Pressure(MPa) |
|---|---|
| 65.2 | 23.23 |
| 59.9 | 22.1 |
| 55.2 | 20.6 |
| 49.8 | 19.1 |
| 45 | 17.6 |
| 40.2 | 15.99 |
| 35.3 | 14.3 |
| 30.4 | 12.65 |
| 25.7 | 10.74 |
| 20.5 | 8.92 |

### EXAMPLE 5

### Dispersion of Particles in Supercritical CO+

### 5.1. Procedure

The properties for stabilization of a dispersion of solid hydrophilic particles are evaluated indirectly in an encapsulation experiment as described in patent EP 0 784 506 B 1.

The CO₂-philic/hydrophilic block copolymer is thus used here as dispersing agent and as coating agent. The experimental device is a device for encapsulation using supercritical CO₂ similar to that used in example 3, equipped with a reactor with an internal volume of 125 ml and with a maximum operating pressure of 30 MPa. The reactor is equipped with optical windows which make it possible to observe the medium at supercritical pressure. 50 mg of spherical particles of ungrafted silica with a diameter of 50 μm (Nucleodur 100-50, Macherey-Nagel) are introduced into the reactor. 200 mg of copolymer P39, described in example 1, are also introduced. The reactor is equipped with a jacket which makes it possible to regulate its temperature. The reactor is closed using a lid equipped with a stirring device of anchor type. Stirring is adjusted to 460 revolutions per minute. Carbon dioxide is introduced into the reactor until a pressure of 20 MPa at a temperature of 45° C. is obtained. Under these conditions, the copolymer is dissolved. A stable and homogeneous dispersion of silica particles in supercritical CO₂ is then observed.

After one hour, the temperature of the reactor is gradually reduced to 20° C. over approximately 50 minutes, so as to bring about the deposition of the copolymer at the surface of the particles. The pressure of the reactor is then reduced to atmospheric pressure over approximately 60 minutes, the carbon dioxide being allowed to escape in a controlled manner by opening a micrometric valve. After opening the reactor, the particles are recovered.

### 5.2. Results

The appearance of the particles recovered after having been subjected to this encapsulation process is presented in FIG. 1.

The starting particles, which have remained separate, have been covered with a homogeneous layer of the copolymer over their entire surface, testifying to the exposure of the whole of their surface to the coating agent. Furthermore, no agglomeration of the particles is recorded. During the use of hydrophobic coating agents, such as, for example, triglycerides, a phenomenon of agglomeration of the particles is commonly observed by the Applicant with the same experimental conditions.

This result demonstrates the ability of the PEO-b-PFDA block copolymer P39 to stabilize a dispersion of hydrophilic particles in CO₂ at supercritical pressure.

### EXAMPLE 6

### Formation of Emulsions in the Presence of Supercritical CO₂

### 6.1. Procedure

The ability of the copolymers described in examples 1 and 2 to stabilize an emulsion is evaluated using a cell for observing phase equilibria at supercritical pressure. This cell is composed of a cylinder made of stainless steel with a maximum operating pressure of 50 MPa and with a volume varying between 50 and 64 ml. The internal volume of the cell is adjusted via a piston actuated by pressurized nitrogen. The temperature of the medium is regulated by circulation of silicone oil in a jacket. The medium can be homogenized using a magnetic bar rotated by magnetic stirring. The cylinder is equipped with two sapphire windows positioned facing one another. The cell is illuminated via the rear window. The medium is observed through the window situated at the front of the cylinder. 22.5 ml of an aqueous solution of a red hydrophilic dye, amaranth, are introduced into the high pressure cylinder. 200 mg of the copolymer P44 are also introduced into the cell. The cylinder is then closed using a lid equipped with a back-pressure piston. The volume is set at 50 ml via the piston. The temperature of the medium is maintained at 40° C. The cylinder is purged using a stream of carbon dioxide and then carbon dioxide is introduced up to a pressure of 30 MPa while maintaining the volume at 50 ml. During the introduction of the carbon dioxide, the white copolymer powder is gradually dissolved. The medium is then stirred and a red and opaque dispersed phase occupying the entire volume of the cylinder is formed.

After 30 minutes, stirring is halted, and slow and gradual phase separation of the system is observed over approximately 5 minutes. A binary system is then observed and does not undergo change over a period of 2 hours. The lower part of the cell is red and opaque. The upper part is red and opalescent. It is an emulsion in CO₂, which are stable without stirring during the two hours of the experiment.

To confirm the role of the copolymer in the formation of this emulsion, the pressure is gradually reduced by gradually raising the piston. Destabilization of the emulsion then occurs and an upper phase becoming colorless and translucent and a lower phase comprising white aggregates, corresponding to the copolymer undissolved because of the reduction in the solvating power of the supercritical dioxide, are observed. An increase in pressure makes it possible to again dissolve the copolymer and to again form a water-in-CO₂ emulsion.

### 6.2. Results

A photograph of the medium seen from the front window of the reactor is represented in FIG. 2. The copolymer has thus made it possible to disperse, in the emulsion form, the aqueous dye solution in the supercritical carbon dioxide at a temperature of 40° C. and a pressure of 30 MPa. An emulsion composed of droplets of the aqueous dye solution in CO₂ is formed.

### EXAMPLE 7

### Comparative Example without Use of Block Copolymers

### 7.1. Procedure

The experimental procedure described in example 6 is used but only the aqueous amaranth solution and the carbon dioxide are introduced into the cell.

### 7.2. Results

In the absence of copolymer and after stirring for one hour, two separate phases are observed in the cell with stirring (FIG. 3). The dense phase is translucent and red and the upper phase is translucent and colorless. Thus, there has been neither formation of emulsion nor of dissolution product of the hydrophilic dye in supercritical carbon dioxide at a temperature of 40° C. and a pressure of 30 MPa.

An identical behavior is observed with a volume of aqueous amaranth solution of 7.5 ml and liquid carbon dioxide at a temperature of 20° C. and a pressure of 20 MPa.

This comparative example confirms the role of the PEO-b-PFDA block copolymer in the formation of an emulsion of water in CO₂ at supercritical pressure.

### EXAMPLE 8

### Formation of an Emulsion in Liquid CO₂ at Supercritical Pressure

A similar experiment to that of example 6 is carried out, except for the temperature, which is set at 20° C., and the volume of aqueous solution, set at 7.5 ml.

At this temperature and under the pressure conditions used, carbon dioxide at supercritical pressure is found in the liquid state. At 20° C. and 30 MPa, an emulsion of water in liquid CO₂ is formed after stirring, as presented in FIG. 4.

The PEO-b-PFDA block copolymer has thus made it possible to form an emulsion of an aqueous solution in liquid CO₂ at supercritical pressure.

### EXAMPLE 9

### Dispersion of Hydrophilic Particles in Supercritical CO₂

The hydrophilic particles used in this example are particles comprising the excipients typically used for formulating lyophilisates comprising therapeutic proteins. The hydrophilic particles are obtained by lyophilization of 100 ml of an aqueous solution comprising 8.39 g of glycine, 410 mg of human serum albumin, 2.4 g of sodium hydrogenphosphate dodecahydrate and 266 mg of sodium dihydrogenphosphate dihydrate. After lyophilization, the particles are ground using an agate mortar and then sieved using a sieve with a mesh size of 25 μm. The sieved particles with a size of less than 25 μm are recovered and used subsequently.

The ability of the copolymers described in examples 1 and 2 to stabilize a dispersion of hydrophilic particles is evaluated using the cell for the observation of phase equilibria described in example 6. 20 mg of hydrophilic particles and 200 mg of PEO-b-PFDA copolymer P44 described in example 2 are introduced into the high pressure cylinder of the observation cell. The temperature of the cylinder is adjusted to 45° C. The volume is set at 50 μl via the backpressure piston. The medium is stirred using a magnetic bar. Carbon dioxide is introduced into the cell until a pressure of 22 MPa is obtained. At this pressure, the copolymer is dissolved and the particles are in suspension in the medium. No particle is observed on the walls of the cylinder or on the sapphire windows. All the hydrophilic particles are therefore dispersed in the stirred medium in the presence of PEO-b-PFDA block copolymer P44. Stirring is then halted. Slow sedimentation of the particles is observed. The sedimentation time, determined by observation of the medium, is 10 minutes in the presence of the PEO-b-PFDA block copolymer P44.

### EXAMPLE 10

### Dispersion of Hydrophilic Particles in Liquid CO₂

An identical experiment is carried out at a temperature of 25° C. and a pressure of 11.5 MPa. When the medium is stirred, all the particles of hydrophilic lyophilisate are dispersed in the liquid CO₂ and no particles are observed on the walls. Stirring is then halted. Slow sedimentation of the particles is observed, with a sedimentation time of the order of 10 minutes.

### EXAMPLE 11

### Comparative Example without Use of Block Copolymer

### 11.1 In Supercritical CO₂

The experimental procedure described in example 9 is followed but only the hydrophilic particles are introduced into the cell before introduction of CO₂ without block copolymer P44. The high pressure cylinder is thermostatically controlled at 45° C. The pressure is increased up to 22 MPa by introduction of CO₂. The medium is stirred via a magnetic bar. It is observed that most of the particles are present on the internal walls of the high pressure cylinder and of the sapphire windows and are not dispersed in supercritical fluid. A few sporadic fine particles are, however, dispersed in the supercritical CO₂. Rapid sedimentation of these sporadic dispersed particles is observed after stirring is halted. The sedimentation time, determined by observing the medium through the sapphire window, is approximately 90 seconds in this case.

11.2. In liquid CO₂

Comparable results are obtained on attempting to disperse these particles in liquid CO₂ according to the procedure described in example 10, at a temperature of 25° C. and at a pressure of 20 MPa. Most of the particles are observed on the walls of the cell and the sapphire windows. A few sporadic particles are dispersed in the liquid CO₂ if the medium is stirred. On halting the stirring, the sedimentation time is approximately 2 minutes.

### 11.3. Conclusion

In the absence of block copolymers, the hydrophilic particles are therefore not effectively dispersed in supercritical or liquid CO₂. Vigorous stirring of the medium makes it possible to disperse only a small proportion of the particles in CO₂ at supercritical pressure, most of the particles being found on the walls of the high pressure cylinder. Stopping the stirring brings about very rapid sedimentation of the sporadic hydrophilic particles dispersed by stirring.

These results, compared with those obtained in examples 9 and 10, confirm that, at 25° C. or 45° C., thus for liquid or supercritical CO₂, the use of PEO-b-PFDA block copolymers makes it possible to efficiently disperse hydrophilic particles in CO₂ at supercritical pressure.

1-28. (canceled)

29. A method for encapsulating an active principle comprising dispersing the active principle in a supercritical fluid by adding a surfactant to the fluid, wherein the surfactant is a block copolymer comprising at least one CO₂-philic block and at least one nonionic hydrophilic block.

30. The method of claim 29, wherein the supercritical fluid is CO₂.

31. The method of claim 29, wherein the supercritical fluid is CO₂ comprising an entrainer in an amount of less than 5%.

32. The method of claim 29, wherein the CO₂-philic block is selected from the group consisting of polymers soluble in supercritical CO₂.

33. The method of claim 29, wherein the block copolymer is a copolymer soluble in supercritical CO₂.

34. The method of claim 33, wherein a minimum solubility of the block copolymer is 0.05% w/w at least one defined temperature between 0° C. and 100° C. and least one defined pressure, that is greater than the supercritical pressure of CO₂ and less than 70 MPa.

35. The method of claim 29, wherein a number-average molar mass of the block copolymer is between 1000 and 200,000 g/mol.

36. The method of claim 35, wherein a number average molar mass of the hydrophilic block is between 500 and 20,000 g/mol.

37. The method of claim 29, wherein a ratio by weight of the CO₂-philic block to the nonionic hydrophilic block is between 1 and 50.

38. The method of claim 29, wherein the CO₂-philic block is selected from the group consisting of fluoropolymers and poly(siloxane)s.

39. The method of claim 38, wherein the fluoropolymers are selected from the group consisting of poly(fluoroether)s, poly(fluoroalkyl acrylate)s and poly(fluoroalkyl methacrylate)s.

40. The method of claim 39, wherein the poly(fluoroalkyl acrylate)s are poly(1,1-dihydroperfluorooctyl acrylate)s or poly(1,1,2,2-tetrahydroperfluorodecyl acrylate)s.

41. The method of claim 29, wherein the nonionic hydrophilic block is selected from biocompatible hydrophilic polymers.

42. The method of claim 29, wherein the biocompatible hydrophilic polymers are selected from the group consisting of polysaccharides, hydrophilic cellulose polymers, poly(vinyl alcohol), polyols and ethylene oxide homo- and copolymers.

43. The method of claim 42, wherein the hydrophilic block is a poly(ethylene oxide).

44. The method of claim 29, wherein the block copolymer is a copolymer composed of a poly(1,1,2,2-tetrahydroperfluorodecyl acrylate)s block and a poly(ethylene oxide) block; a PEO-b-PFDA block copolymer; a PFDA-b-PEO-b-PFDA triblock copolymer or a PEO-b-PFDA-b-PEO triblock copolymer.

45. The method of claim 29, wherein the water-soluble or hydrophilic substance comprise an active principle, wherein the active principle is selected from the group consisting of (i) pharmaceuticals; (ii) cosmetics; and (iii) foodstuffs.

46. The method of claim 45, wherein the pharmaceuticals are selected from the group consisting of analgesics, antipyretics, aspirin and aspirin derivatives, antibiotics, antiinlammatories, antiulceratives, antihypertensives, neuroleptics, antidepressants, therapeutic oligonucleotides, therapeutic peptides and therapeutic proteins.

47. The method of claim 46, wherein the therapeutic peptides and the therapeutic proteins are selected from the group consisting of a protein corresponding to parathyroid hormone, growth hormone, α-interferons, β-interferons, γ-interferons, α-erythropoietin, β-erythropoietin, granulocyte colony-stimulating factor, granulocyte-macrophage colony-stimulating factor, vasoactive intestinal peptide, thyrotopine-releasing hormone, arginine vasopressin, angiotensin, insulin, somatotropin, tissue plasminogen activator, clotting factors VIII and IX, glucosylceramidase, lenograstim, molgramostim, filgrastim, interleukins, dornase alfa, PEG-L-asparaginase, PEG-adenosine deaminase, hirudin, eptacog alfa, nerve growing factors, luteinizing hormone-releasing hormone and its derivatives and analogs, somatostatin and its derivatives, triptorelin, bombesin, calcitonin, gastrin-releasing peptide, growth hormone-releasing factor and amylin.
